(19) United States
(12) Patent Application Publication
(10) Pub. No.: US 2022/0267718 A1
(43) Pub. Date: Aug. 25, 2022

(54) BACTERIAL-BASED MODULATION OF PLANT GROWTH

(71) Applicant: AGROWORLD PRECISION AGRICULTURE INC., DOLLARD-DES-ORMEAUX (CA)

(72) Inventors: DONALD L. SMITH, SAINTE-ANNE-DE-BELLEVUE (CA); GAYATHRI ILANGUMARAN, SAINTE-ANNE-DE-BELLEVUE (CA)

(21) Appl. No.: 17/626,297

(22) PCT Filed: Jul. 10, 2020
(86) PCT No.: PCT/CA2020/050959

§ 371 Date: Jan. 11, 2022

Related U.S. Application Data
(60) Provisional application No. 62/873,304, filed on Jul. 12, 2019.

(30) Foreign Application Priority Data
Jul. 12, 2019 (CA) 3049530

(51) Int. Cl. C12N 1/20; A01N 63/20; C05F 11/08

(52) U.S. Cl. CPC C12N 1/205; A01N 63/20; C05F 11/08; C12R 2001/01

(57) ABSTRACT

Disclosed herein are bacterial strains and products thereof, and uses thereof for example to increase, improve or facilitate plant growth. Also disclosed herein are method to increase or improve plant tolerance or resistance to one or more abiotic stress conditions.

### CROSS REFERENCE TO RELATED APPLICATIONS

The present application claims the benefit of U.S. Provisional application No. 62/873,304 filed Jul. 12, 2019, which is incorporated herein by reference.

### TECHNICAL FIELD

The present disclosure generally relates to the modulation of plant growth, in particular using bacteria and/or products thereof.

### REFERENCE TO DEPOSITS OF BIOLOGICAL MATERIAL

This application contains references to deposits of biological material, which deposits are incorporated herein by reference.

### BACKGROUND ART

Agricultural crops and productivity are of great importance. For example, soybean is the most widely grown legume in the world, valued for its high protein and oil content in the seeds¹. It is the fourth largest field crop seeded in Canada (7.7 million tonnes produced in 2017)² while in the Unites States, it is the second largest crop (119.5 million tonnes produced in 2017)³ second only to corn.

Plants are sensitive to various abiotic stresses. For example, soybean is a glycophyte and is sensitive to salinity; salinity stress inhibits seed germination and seedling growth, reduces pod number and nodulation, decreases shoot biomass and seed weight; salt-affected soils can decrease soybean yield up to 40%^{4,5,6}. Dryland salinity has dramatically affected agriculture in the Rocky Mountains, the Prairies and the Northern Great Plains^{7,8}.

There is thus a need for alternative materials and methods to improve plant growth, and also for example to improve plant tolerance/resistance to abiotic stresses, including for example improving plant salt tolerance under conditions of salinity stress.

The present description refers to a number of documents, the content of which is herein incorporated by reference in their entirety.

### SUMMARY OF THE DISCLOSURE

The present disclosure generally relates to bacterial strains and products thereof, and uses thereof for example to increase, improve or facilitate plant growth.

In various aspects and embodiments, the present disclosure provides the following items:

1. A biologically pure culture of a bacterial strain selected from a bacterial strain comprising substantially all of the biochemical characteristics of bacterial strain AB2 deposited at the IDAC under accession no. 090719-01 on Jul. 9, 2019 or a mutant thereof comprising substantially all of the biochemical characteristics thereof; a bacterial strain comprising substantially all of the biochemical characteristics of bacterial strain AB3 deposited at the IDAC under accession no. 090719-02 on Jul. 9, 2019 or a mutant thereof comprising substantially all of the biochemical characteristics thereof; and a bacterial strain comprising substantially all of the biochemical characteristics of bacterial strain AB8 deposited at the IDAC under accession no. 090719-03 on Jul. 9, 2019 or a mutant thereof comprising substantially all of the biochemical characteristics thereof.
2. The biologically pure culture of a bacterial strain of item 1, wherein the bacterial strain is a bacterial strain comprising substantially all of the biochemical characteristics of bacterial strain AB2 deposited at the IDAC under accession no. 090719-01 on Jul. 9, 2019 or a mutant thereof comprising substantially all of the biochemical characteristics thereof.
3. The biologically pure culture of a bacterial strain of item 1, wherein the bacterial strain is a bacterial strain comprising substantially all of the biochemical characteristics of bacterial strain AB3 deposited at the IDAC under accession no. 090719-02 on Jul. 9, 2019 or a mutant thereof comprising substantially all of the biochemical characteristics thereof.
4. The biologically pure culture of a bacterial strain of item 1, wherein the bacterial strain is a bacterial strain comprising substantially all of the biochemical characteristics of bacterial strain AB8 deposited at the IDAC under accession no. 090719-03 on Jul. 9, 2019 or a mutant thereof comprising substantially all of the biochemical characteristics thereof.
5. A biologically pure culture of a bacterial strain selected from bacterial strain AB2 deposited at the IDAC under accession no. 090719-01 on Jul. 9, 2019 or a mutant thereof comprising substantially all of the biochemical characteristics thereof; bacterial strain AB3 deposited at the IDAC under accession no. 090719-02 on Jul. 9, 2019 or a mutant thereof comprising substantially all of the biochemical characteristics thereof; and bacterial strain AB8 deposited at the IDAC under accession no. 090719-03 on Jul. 9, 2019 or a mutant thereof comprising substantially all of the biochemical characteristics thereof.
5.1 A biologically pure culture of a bacterial strain selected from bacterial strain AB2 deposited at the IDAC under accession no. 090719-01 on Jul. 9, 2019 or a mutant thereof comprising a 16s rRNA sequence that is substantially identical to a 16s rRNA sequence of bacterial strain AB2; bacterial strain AB3 deposited at the IDAC under accession no. 090719-02 on Jul. 9, 2019 or a mutant thereof comprising a 16s rRNA sequence that is substantially identical to a 16s rRNA sequence of bacterial strain AB3; and bacterial strain AB8 deposited at the IDAC under accession no. 090719-03 on Jul. 9, 2019 or a mutant thereof comprising a 16s rRNA sequence that is substantially identical to a 16s rRNA sequence of bacterial strain AB8.
6. The biologically pure culture of a bacterial strain of item 5, wherein the bacterial strain is bacterial strain AB2 deposited at the IDAC under accession no. 090719-01 on Jul. 9, 2019 or a mutant thereof comprising substantially all of the biochemical characteristics thereof.
6.1 The biologically pure culture of a bacterial strain of item 5, wherein the bacterial strain is bacterial strain AB2 deposited at the IDAC under accession no. 090719-01 on Jul. 9, 2019 or a mutant thereof comprising a 16s rRNA sequence that is substantially identical to a 16s rRNA sequence of bacterial strain AB2.
7. The biologically pure culture of a bacterial strain of item 5, wherein the bacterial strain is bacterial strain AB3 deposited at the IDAC under accession no. 090719-02 on Jul. 9, 2019 or a mutant thereof comprising substantially all of the biochemical characteristics thereof.
7.1 The biologically pure culture of a bacterial strain of item 5, wherein the bacterial strain is bacterial strain AB3 deposited at the IDAC under accession no. 090719-02 on Jul. 9, 2019 or a mutant thereof comprising a 16s rRNA sequence that is substantially identical to a 16s rRNA sequence of bacterial strain AB3.
8. The biologically pure culture of a bacterial strain of item 5, wherein the bacterial strain bacterial strain AB8 deposited at the IDAC under accession no. 090719-03 on Jul. 9, 2019 or a mutant thereof comprising substantially all of the biochemical characteristics thereof.
8.1 The biologically pure culture of a bacterial strain of item 5, wherein the bacterial strain is bacterial strain AB8 deposited at the IDAC under accession no. 090719-03 on Jul. 9, 2019 or a mutant thereof comprising a 16s rRNA sequence that is substantially identical to a 16s rRNA sequence of bacterial strain AB8.
9. A culture product of the bacterial strain defined in any one of items 1 to 8.
9.1 The culture product of item 9, which is a substantially cell-free supernatant or extract of the bacterial strain.
9.2 The culture product of item 9, prepared by a process comprising centrifugation and/or filtration to substantially remove the bacterial cells.
10. A composition comprising the bacterial strain defined in any one of items 1 to 8 and/or the culture product of item 9 and a carrier.
11. A method comprising applying the bacterial strain defined in any one of items 1 to 8, the culture product of item 9, and/or the composition of item 10, to a plant or part thereof, a seed of a plant, and/or an area around the seed, plant or part thereof.
12. A method for increasing a plant's growth, comprising applying the bacterial strain defined in any one of items 1 to 8, the culture product of item 9, and/or the composition of item 10, to a plant or part thereof, a seed of a plant, and/or an area around the seed, plant or part thereof, in an amount effective to produce an increase in plant growth as compared to the growth of the plant in the absence of said application of said bacterial strain, culture product and/or composition.
13. The method of item 11 or 12, wherein the plant or seed is for growth under abiotic stress conditions.
14. The method of item 13, wherein the abiotic stress conditions are selected from high salinity, drought, high temperature, low temperature and flooding.
15. The method of item 14, wherein the abiotic stress conditions comprise high salinity.
16. A method for increasing tolerance of a plant or seed to one or more abiotic stress conditions, comprising applying the bacterial strain defined in any one of items 1 to 8, the culture product of item 9, and/or the composition of item 10, to the plant or part thereof, the seed, and/or an area around the seed, plant or part thereof, in an amount effective to produce an increase in tolerance to the one or more abiotic stress conditions as compared to the tolerance of the plant in the absence of said application of said bacterial strain, culture product and/or composition.
17. The method of item 16, wherein the abiotic stress conditions are selected from high salinity, drought, high temperature, low temperature and flooding.
18. The method of item 17, wherein the abiotic stress conditions comprise high salinity.
19. The method of any one of items 11 to 18, wherein the plant is a leguminous plant.
20. The method of item 19, wherein the plant is soybean.
21. The composition of item 10, which is a seed coating composition.
22. A seed partially or completely coated with the bacterial strain defined in any one of items 1 to 8, the culture product of item 9, and/or the composition of item 10 or 21.
23. A method of preparing the culture product of item 9, comprising culturing the bacterial strain defined in any one of items 1 to 8 and recovering the culture product from the culture.
24. A kit comprising the bacterial strain defined in any one of items 1 to 8, the culture product of item 9, and/or the composition of item 10.
25. The kit of item 24, further comprising instructions for use of the bacterial strain, culture product, and/or composition.

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1: Crude supernatant from AB3 culture promotes root growth of Arabidopsis under salt stress (bottom).

FIG. 2: Bacterized soybean seed germination % under optimal and salt conditions from 24 to 48 h (n=6). 24 h, bottom section of bars; 36 h, middle section of bars; 48 h, upper section of bars.

FIG. 3: Soybean shoot biomass under optimal and salt stress conditions. Soybean seeds treated with isolated bacteria and plants were sampled after 4 weeks of growth (n=6; * indicates difference from the control at p<0.1 indicates difference from the control at p<0.05).

FIG. 4: Soybean plants at early vegetative stage. Bacteria-treated plants show fully emerged second trifoliate under optimal and salt stress conditions.

FIG. 5: Leaf area and shoot biomass of soybean plants treated with PGPR strains AB2 and AB8 after 4 weeks of growth (n=6; * indicates difference from the control at p<0.1 ** indicates difference from the control at p<0.05). In each group of bars: left bar, Ctrl; middle bar, AB2; right bar, AB8.

FIG. 6: Root length of Arabidopsis plants measured using Image J. Extract was added to the growth media under optimal and salt stress conditions. Yeast extract mannitol (YEM) was used as a negative control and values that are indicated with S are from salt stressed plants (n=6; *** indicates difference from the control at p<0.001).

### DETAILED DESCRIPTION

### General Definitions

The use of the terms “a” and “an” and “the” and similar referents in the context of describing the subject matter (especially in the context of the following claims) are to be construed to cover both the singular and the plural, unless otherwise indicated herein or clearly contradicted by context.

The use of the word “a” or “an” when used in conjunction with the term “comprising” in the claims and/or the specification may mean “one” but it is also consistent with the meaning of “one or more”, “at least one”, and “one or more than one”.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended (i.e., meaning “including, but not limited to”) and do not exclude additional, un-recited elements or method steps.

As used herein, the term “consists of” or “consisting of” means including only the elements, steps, or ingredients specifically recited in the particular claimed embodiment or claim.

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

Herein, the term “about” has its ordinary meaning. The term “about” is used to indicate that a value includes an inherent variation of error for the device or the method being employed to determine the value. For example, the terminology “about” is meant to designate a possible variation of up to 10%, i.e. within 10% of the recited values (or range of values). Therefore, a variation of 1, 2, 3, 4, 5, 6, 7, 8, 9 and 10% of a value is included in the term “about”. Unless indicated otherwise, use of the term “about” before a range applies to both ends of the range.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Similarly, herein a general chemical structure with various substituents and various radicals enumerated for these substituents is intended to serve as a shorthand method of referring individually to each and every molecule obtained by the combination of any of the radicals for any of the substituents. Each individual molecule is incorporated into the specification as if it were individually recited herein. Further, all subsets of molecules within the general chemical structures are also incorporated into the specification as if they were individually recited herein.

All methods described herein can be performed in any suitable order unless otherwise indicated herein or otherwise clearly contradicted by context.

The use of any and all examples, or exemplary language (“e.g.”, “such as”) provided herein, is intended merely to better illustrate the disclosure and does not pose a limitation on the scope of the disclosure unless otherwise claimed.

Terms and symbols of genetics, molecular biology, biochemistry and nucleic acid used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like. All terms are to be understood with their typical meanings established in the relevant art.

Unless otherwise defined, all technical and scientific terms used herein have the same meaning as commonly understood by one of ordinary skill in the art to which this disclosure belongs.

### Bacterial Strains and Uses Thereof

In the studies described herein, bacterial strains have been isolated from the root system of a native legume (Amphicarpaea bracteata, American Hog peanut) and screened for ability to promote plant growth, including characterizing their beneficial effects in soybean under stressed (salt stress) and unstressed conditions, in order to determine the capacity for crop plant growth promotion and enhancement of stress tolerance. We have generally found that resistance to one abiotic stress indicates resistance to most or all of the stressors; given the significant overlap in plant responses to abiotic stresses (e.g., salt, water deficit, heat, cold, flooding).

Out of the strains isolated, three novel strains were identified as particularly interesting. Two of these isolates (AB2 and AB8) caused increases (greater than 60%) in the germination rate of soybean seeds (seeds treated with bacterial suspension and held on moist filter paper in a petri plate), and particularly under stressful conditions. In this case the evaluation was under salt stress (100 mM NaCl), which is a challenging level of stress for soybean. Salt stress is an easy type of stress to apply uniformly under research conditions. These same two strains were found to enhance the growth of soybean plants through the early vegetative stages when plants were under salt stress (again 100 mM NaCl). The increases in growth, averaged over replicates, were close to 30%. These same two strains also caused increases in the growth of unstressed soybean plants. The supernatant (cell-free extract) of a third strain (AB3) caused dramatic increases in the growth of Arabidopsis roots when the plant was under stressful conditions, and was also shown to increase seed germination in a variety of crops (canola, soybean, corn, chili pepper, Chinese celery, eggplant and tomato). This strain produces compound(s) that are excreted into the growth medium so that liquid growth medium has the ability to promote plant root growth after the strain has been grown in it, and the cells then removed.

These studies have allowed the identification and characterization of new plant growth promoting rhizobacteria (PGPR) strains isolated from a native plant species. The ability to increase plant growth and biomass production, under both stressed and unstressed conditions, has been established for the isolated strains (e.g. AB2 and AB8). Also observed was the plant growth promoting activity of crude extracts (e.g. AB3). These novel PGPR strains can be effectively applied as an agriculture input to improve crop yields under abiotic stress conditions.

In various aspects, the present disclosure concerns a biologically pure culture of a bacterial strain, e.g. a rhizobial strain, and/or materials/products produced by and/or excreted by such a strain, and uses thereof for example to increase, improve or facilitate plant growth.

In embodiments, disclosed herein is a biologically pure culture of a bacterial strain isolated from an undomesticated local legume species. In embodiments, disclosed herein is a biologically pure culture of a bacterial strain comprising substantially all of the biochemical characteristics of bacterial strain AB2 deposited at the IDAC under accession no. 090719-01 on Jul. 9, 2019 or a mutant thereof comprising substantially all of the biochemical characteristics thereof; a bacterial strain comprising substantially all of the biochemical characteristics of bacterial strain AB3 deposited at the IDAC under accession no. 090719-02 on Jul. 9, 2019 or a mutant thereof comprising substantially all of the biochemical characteristics thereof; and a bacterial strain comprising substantially all of the biochemical characteristics of bacterial strain AB8 deposited at the IDAC under accession no. 090719-03 on Jul. 9, 2019 or a mutant thereof comprising substantially all of the biochemical characteristics thereof.

In embodiments, disclosed herein is a biologically pure culture of a bacterial strain selected from bacterial strain AB2 deposited at the IDAC under accession no. 090719-01 on Jul. 9, 2019 or a mutant thereof comprising substantially all of the biochemical characteristics thereof; bacterial strain AB3 deposited at the IDAC under accession no. 090719-02 on Jul. 9, 2019 or a mutant thereof comprising substantially all of the biochemical characteristics thereof; and bacterial strain AB8 deposited at the IDAC under accession no. 090719-03 on Jul. 9, 2019 or a mutant thereof comprising substantially all of the biochemical characteristics thereof.

16S rRNA gene sequences contain hypervariable regions that can provide species-specific signature sequences useful for identification of bacteria. In embodiments, disclosed herein is a biologically pure culture of a bacterial strain selected from bacterial strain AB2 deposited at the IDAC under accession no. 090719-01 on Jul. 9, 2019 or a mutant thereof comprising a 16s rRNA sequence that is substantially identical to a 16s rRNA sequence of bacterial strain AB2; bacterial strain AB3 deposited at the IDAC under accession no. 090719-02 on Jul. 9, 2019 or a mutant thereof comprising a 16s rRNA sequence that is substantially identical to a 16s rRNA sequence of bacterial strain AB3; and bacterial strain AB8 deposited at the IDAC under accession no. 090719-03 on Jul. 9, 2019 or a mutant thereof comprising a 16s rRNA sequence that is substantially identical to a 16s rRNA sequence of bacterial strain AB8.

“Substantially identical” as used herein refers to nucleic acids, e.g., RNAs, having at least 60% of similarity, in embodiments at least 65%, 70%, 75%, 80%, 85%, 90%, 91%, 92%, 93%, 94%, 95%, 96%, 97%, 98% or 99% of similarity in their nucleotide sequences. In further embodiments, the nucleic acids have at least 60%, 70%, 75%, 80%, 85%, 90%, 91%, 92%, 93%, 94%, 95%, 96%, 97%, 98% or 99% of identity in their nucleotide sequences. Optimal alignment of sequences for comparisons of identity may be conducted using a variety of algorithms, such as the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman (Pearson and Lipman 1988), and the computerized implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al. (Altschul et al. 1990) 1990 (using the published default settings). Software for performing BLAST analysis may be available through the National Center for Biotechnology Information (through the internet at http://www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighborhood word score threshold. Initial neighborhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. One measure of the statistical similarity between two sequences using the BLAST algorithm is the smallest sum probability (P(N)), which provides an indication of the probability by which a match between two nucleotide or amino acid sequences would occur by chance. In alternative embodiments of the disclosure, nucleotide or amino acid sequences are considered substantially identical if the smallest sum probability in a comparison of the test sequences is less than about 1, preferably less than about 0.1, more preferably less than about 0.01, and most preferably less than about 0.001.

Also disclosed herein is a culture product of a bacterial strain described herein, as well as a composition comprising a bacterial strain described herein and/or a culture product thereof and a carrier (e.g. an agriculturally acceptable carrier). Also disclosed herein is an inoculum comprising a bacterial strain described herein. In embodiments the composition or inoculum comprise two or more of the bacterial strains described herein.

In embodiments, a bacterial strain, culture product and/or composition described herein may be applied to a plant, a plant part, a seed and/or an area around the seed, plant or part thereof. Therefore, also disclosed is a method comprising applying a bacterial strain, culture product and/or composition described herein to a plant or part thereof, a seed of a plant, and/or an area around the seed, plant or part thereof. As used herein the terms “area around the seed, plant or part thereof” refers to the plant substrate (e.g., soil, etc.) prior to planting the plant seedling or seed or after having planted the plant seedling or seed, it includes such substrates prior to planting as well as after planting/during plant growth in the substrate. In embodiments, such methods and uses are for increasing, improving or facilitating plant growth.

In embodiments, the plant or seed is for growth under abiotic stress conditions, and the method or use is for increasing or improving tolerance or resistance of the plant or seed to such abiotic stress conditions (e.g., high salinity, drought, high temperature, low temperature and flooding).

In embodiments, a bacterial strain, culture product and/or composition described herein may be used in the form of a seed coating. Thus also disclosed herein is a seed partially or completely coated with a bacterial strain, culture product and/or composition described herein.

Also disclosed is a kit or package comprising a bacterial strain, culture product and/or composition described herein. In embodiments, the kit or package may further comprise instructions for use of a bacterial strain, culture product and/or composition described herein, e.g. for increasing, improving or facilitating plant growth, and/or for increasing or improving tolerance or resistance of the plant or seed to such abiotic stress conditions. Such a kit or package may in embodiments comprise one or more containers for the component(s) thereof, as well as e.g. growth/culture media or suitable carriers or diluents for use of the bacterial strain, culture product and/or composition.

“Plant growth” means all or part of the process that begins with a plant seed and continues to a mature plant. Plant growth includes seed germination, the plant emerging from the soil, the formation of roots, stems and leaves, as well as an increase in size and mass of the plant. Plant growth may be determined for example by rate or percentage of germination, biomass, rate of growth, development of leaves/foliate structures, leaf area, root number, root length, root mass, etc. In embodiments, “plant growth” refers to (i) an increase in the number of leaves in the plant; (ii) an increased in the plant's height; (iii) an increase in the root and/or shoot biomass; (iv) an increase in seed yield/number; (v) an increase in the total tiller number; (vi) an increased ratio of reproductive tiller/total tiller; (vii) an increase in chlorophyll content leading to darker leaves; (viii) an increase in field forage and grain crop yield; (ix) an increase in crop quality, (x) an increase in the rate of germination; (xi) an increase in frequency of germination; or (xii) a combination of at least two of (i) to (xi). In embodiments, the term “increasing” in the expression “increasing plant growth” refers to an increase of one or more of the above characteristics of at least about 2% as compared to a reference plant growth (e.g., that of the plant in the absence of a bacterium or culture product thereof described herein). In an embodiment, the increase of one or more of the above characteristics is of at least about 4%, in a further embodiment, at least about 5%, in a further embodiment, at least about 6%, in a further embodiment, at least about 8%, in a further embodiment, at least about 10%, in a further embodiment, at least about 12%, in a further embodiment, at least about 15%, in a further embodiment, at least about 20%, in a further embodiment of at least about 30%, in a further embodiment of at least about 40%, in a further embodiment at least about 50%, in a further embodiment of at least about 60%, in a further embodiment of at least about 70%, in a further embodiment of at least about 80%, in a further embodiment of at least about 90%, in a further embodiment of about 100%.

“Plant substrate” refers to a substrate or material used for growing plants, including seeds, plant roots and plant seedlings. Such plant substrates include for example soil, peat, compost, vermiculite, perlite, sand, clay, and combinations thereof. Other types of plant substrates are also included, depending on the plant growth system/environment being used.

“Growing a plant” as used herein means to place a plant (e.g., seed, seedling, mature plant) in a location/substrate and under conditions suitable for plant growth.

“Abiotic stress conditions” as used herein refer to conditions caused by non-living factors on plants in a particular environment, which may inhibit plant growth or maintenance. Examples of abiotic stress conditions include high salinity, drought, high temperature, low temperature and freezing.

The ions involved in soil salinity include Na⁺, K⁺, Ca²⁺, Mg²⁺ and Cl⁻; since sodium often predominates, soils can also be referred to as sodic. Soil salinity is typically measured as the salt concentration in the water extracted from saturated soil (referred to as the saturation extract). High salinity typically refers to at least about 75 mM, 80 mM, 85 mM, 90 mM, 95 mM or 100 mM, 110 mM, 120 mM, 130 mM, 140 mM, 150 mM, 175 mM or 200 mM salt (e.g., NaCl), in a further embodiment at least about 100 mM salt (e.g., NaCl), in embodiments about 100 to about 300 mM salt (e.g., NaCl), or in other embodiments, higher. In embodiments, high salinity refers to at least about 3, 4, 5, 6, 7, 8, 9, 10, 11 or 12 g/I salt. Salinity occurs through natural processes (e.g. weathering of rocks/minerals; salt from the ocean; salt accumulation in dry regions) or human-induced processes (e.g., irrigation, road salt) that result in the accumulation of salts in the soil to a level that inhibits plant growth. High temperature is an increase above ambient temperature, e.g. at least about 5, 10 or 15° C. above ambient temperature. Other factors in heat stress relate to e.g. the duration of the elevated temperature and rate of increase in temperature.

“Drought conditions” as used herein refer to the set of environmental conditions under which a plant will begin to suffer the effects of water deprivation, such as decreased stomatal conductance and photosynthesis, decreased growth rate, loss of turgor (wilting), significant reduction in biomass production and crop yield, or increased ovule abortion. Plants experiencing drought stress typically exhibit a significant reduction in biomass and yield. Water deprivation may be caused by lack of rainfall or limited irrigation. Alternatively, water deficit may also be caused by high temperatures, low humidity, saline soils, freezing temperatures or water-logged soils that damage roots and limit water uptake to the shoot. Since plant species vary in their capacity to tolerate water deficit, the precise environmental conditions that cause drought stress cannot be generalized. Limited availability of water or drought is to be understood as a situation wherein water is or may become a limiting factor for biomass accumulation or crop yield for a non-drought resistant plant.

Growing a plant under abiotic stress conditions refers to growing a plant under conditions whereby during its growth, it is exposed to such conditions for at least a period of time.

“Tolerant” or “tolerance” generally refers to an ability to live, grow and/or function under one or more adverse conditions.

“Increase” or “improve” as used herein in relation to plant growth in the context of a particular treatment (e.g., with a bacterial strain described herein or a culture product thereof), means that plant growth is generally improved for one or more factors or properties as compared to a standard or control lacking the treatment. Similarly, “increase” or “improve” as used herein in relation to seed germination in the context of a particular treatment (e.g., with a bacterial strain described herein or a culture product thereof) means that seed germination is improved (e.g., faster (at an increased rate), increase in percentage of seeds that germinate or a greater proportion of seeds germinate overall) as compared to a standard or control lacking the treatment. Increasing or improving seed germination is encompassed by the terms increasing or improving plant growth (i.e., a treatment or component that increases or improves seed germination also increases or improves plant growth; a treatment or component that increases or improves plant growth may not necessarily facilitate seed germination).

“Increase” or “improve” as used herein in relation to plant tolerance or resistance to one or more abiotic stress conditions in the context of a particular treatment (e.g., with a bacterial strain described herein or a culture product thereof) relates to an increase in tolerance or resistance to the one or more abiotic stress conditions as compared to the tolerance or resistance of the plant in the absence of said application of said bacterial strain, culture product and/or composition. In embodiments, such an increase or improvement in tolerance or resistance to one or more abiotic stress conditions, may be a decrease of the inhibition, reduction in inhibition of plant growth or a decrease in crop failure under such abiotic stress condition(s). In embodiments, such an increase or improvement in tolerance or resistance is at least a 10%, 20%, 30%, 40%, 50%, 60%, 70%, 80%, or 90% decrease of the inhibition, reduction of plant growth or decrease in crop failure under such abiotic stress condition(s), in a further embodiment, a 100% decrease of the inhibition, reduction or plant growth or decrease in crop failure under such abiotic stress condition(s).

A plant may include the entirety of a plant or may include one or more forms and/or parts thereof, above or below ground, such as shoots, leaves, flowers, roots, needles, stalks, stems, fruit bodies, fruits, seeds, roots, tubers, and rhizomes, and the like. Plants may also include harvested material and vegetative and generative propagation material (e.g., seeds, cuttings, tubers, rhizomes, off-shoots, seeds).

In embodiments, a bacterium described herein can be a rhizobium or rhizobial bacterium. Such bacteria are generally from the soil and can fix nitrogen and provide it in forms usable by plants, e.g. by forming nodules on or within the roots of plants. In embodiments, the rhizobial bacterium is of the family Bradyrhizobiaceae, Brucellaceae, Hyphomicrobiaceae, Methylobacteriaceae, Phyllobacteriaceae, Rhizobiaceae or Burkholderiaceae. There are a number of different genera of bacteria that are within the rhizobium grouping. One genus of organisms therein includes Bradyrhizobium.

### Forms and Administration/Application of the Bacterial Strains Described Herein

“Biologically pure” as used herein in the context of a bacterial strain refers to a strain separated from materials with which it is normally associated in nature. Note that a strain associated with compounds or materials that it is not normally found with in nature, is still defined as “biologically pure”. For example, a strain associated with laboratory culture media is considered to be “biologically pure”. A monoculture of a particular strain is, of course, “biologically pure.”

For the methods and uses of the present invention, it is not necessary that the whole broth culture of the strains be used. Indeed, the present disclosure encompasses the use of a whole broth culture of a strain described herein, dried biomass of the strains and lyophilized strains. As used herein therefore, application of a strain described herein refers to application of any form or part of the strain described herein.

Also disclosed herein are culture products of bacterial strains described herein. A “culture product” refers to any kind of material associated with or generated by a bacterial strain, e.g., one or more compounds or a solution or mixture present in the growth medium in which the bacterial strain is cultured. Such culture products may in embodiments be used in the form of the culture comprising bacterial cells, or in embodiments may be used in an isolated or purified form obtained for example by centrifugation and/or filtration to substantially remove bacterial cells (i.e., in substantially cell-free form). Such culture products are sometimes referred to as a “medium”, “broth”, “supernatant”, “extract”, “cell-free supernatant”, “cell-free extract” or “filtrate.” In embodiments, such culture products may be used in undiluted or diluted form, e.g., diluted at least 10, 25, 50, 75, 100, 150, 200, 250, 300, 350, 400, 450, 500, 550, 600, 650, 700, 750, 800, 850, 900, 950, 1000, 1100, 1200, 1300, 1400 or 1500-fold. In embodiments, the culture product, in undiluted or diluted form, may be applied to crops in an amount of at least about 0.1, 1, 5, 7.5 or 10 I/acre, e.g., at least about 0.1, 1, 5, 7.5 or 10 l of a 10, 25, 50, 75 or 100-fold dilution of the culture product per acre. In embodiments, the culture product, in undiluted or diluted form, may be applied to crops in an amount of at least about 0.2, 1, 2.5, 5.0, 7.5, 10, 12.5, 15, 20 or 25 I/hectare, e.g., at least about 0.2, 1, 2.5, 5.0, 7.5, 10, 12.5, 15, 20 or 25 l of a 10, 25, 50, 75 or 100-fold dilution of the culture product per hectare.

Also disclosed herein is a method of preparing a culture product described herein, comprising culturing a bacterial strain described herein and recovering or isolating the culture product from the culture (e.g. by substantial removal of bacterial cells, for example by centrifugation and/or filtration).

A bacterial strain described herein, a culture product thereof, and/or a composition thereof may be applied to soil directly prior to seeding the plant or after planting the plant, sprayed on the plant, soil and/or on the seed of the plant. Said seed may be applied to soil directly. “Applying”, “apply”, “applied” or “application” when used in the context of applying a bacterial strain described herein, or a culture product thereof or a composition thereof, to a plant or part thereof or a seed, means placing the bacterial strain described herein, or a culture product thereof or a composition thereof in close enough proximity to the plant, plant part and/or seed so that the bacterial strain or a substance produced by the strain, the culture product or composition is capable of facilitating or enhancing growth of the plant, directly and/or indirectly. Such application may be directly to the plant or part thereof or seed, or to the environment/substrate (e.g. soil) in which the plant or seed will be grown. In embodiments, such application may occur before (e.g. to a furrow prior to planting) or after planting, or during growth of more mature plants.

Also disclosed is a combination of a bacterial strain or culture product thereof described herein and one or more carriers to form a composition. Formulating such a composition may increase potential storage time and stability. In embodiments, such a composition may further comprise other components for improving or facilitation plant growth, such as other bacterial strains, fertilizers, pesticides, etc.

In order to achieve good dispersion, adhesion and conservation/stability of compositions within the present invention, it may be advantageous to formulate a bacterial strain or culture product thereof described herein with components that aid dispersion, adhesion and conservation/stability or even assist in the tolerance/resistance of the plant on which it is applied (e.g., in the context of an abiotic stress condition). It could be formulated as a spray, or as a coating for the plant seed. These components are referred to herein individually or collectively as “carrier”. Suitable formulations for this carrier will be known to those skilled in the art (wettable powders, granules and the like, or carriers within which the inoculum can be microencapsulated in a suitable medium and the like, liquids such as aqueous flowables and aqueous suspensions, and emulsifiable concentrates).

For example, peat-based inoculant represents a widely used type of formulation. Alternative methods such as the encapsulation of a microorganism with a biopolymer are also encompassed. Encapsulation is the process of making a protective capsule around the microorganism. The matrix of microsphere protects the cells by providing pre-defined and constant microenvironment thus allowing the cells to survive and maintain metabolic activity for extended periods of time. Microspheres can provide a controlled release of microorganisms as well as serve as energy source for the microorganism, due to its degradation. Different natural polysaccharides and protein co-extruded with calcium alginate in order to form a gelled matrix, matrix material such as starches, maltodextrin, gum Arabic, pectin, chitosan, alginate and legumes protein are also encompassed by the present invention (Khan, Korber et al. 2013, Nesterenko, Alric et al. 2013). Without being so limited, useful carriers for the present invention include propylene glycol alginate, powder or granular inert materials may include plant growth media or matrices, such as rockwool and peat-based mixes, attapulgite clays, kaolinic clay, mont-morillonites, saponites, mica, perlites, vermiculite, talc, carbonates, sulfates, oxides (silicon oxides), diatomites, phytoproducts, (ground grains, pulses flour, grain bran, wood pulp, and lignin), synthetic silicates (precipitated hydrated calcium silicates and silicon dioxides, organics), polysaccharides (gums, starches, seaweed extracts, alginates, plant extracts, microbial gums), and derivatives of polysaccharides, proteins, such as gelatin, casein, and synthetic polymers, such as polyvinyl alcohols, polyvinyl pyrrolidone, polyacrylates (Date and Roughley, 1977; Dairiki and Hashimoto, 2005; Jung et al., 1982). The carrier may include components such as chitosan, vermiculite, compost, talc, milk powder, gel, etc. Other suitable formulations will be known to those skilled in the art.

As used herein, the terminology “amount effective” or “effective amount” is meant to refer to an amount sufficient to effect beneficial or desired results. An effective amount can be provided in one or more administrations. For example, in terms of inducing tolerance or resistance to high salinity in a plant, an “effective amount” of a microorganism (e.g. bacterial strain) or a microorganism product (e.g. culture product) described herein is an amount sufficient to increase tolerance/resistance to high salinity in a plant as compared to that exhibited by plant in the absence of the microorganism or microorganism product. In a further example, in terms of inducing drought tolerance/resistance in plant, an “effective amount” of microorganism (e.g. bacterial strain) or a microorganism product (e.g. culture product) is an amount sufficient to increase drought tolerance/resistance in a plant as compared to that exhibited by plant in the absence of the microorganism or microorganism product. In a specific embodiment, it refers to an amount of about 1×10⁸ CFU or more/plant, plant part, or area around a plant or plant part.

### Plants

In embodiments, the methods and systems described herein may be used for a variety of plants, including monocotyledonous and dicotyledonous plants. In embodiments the plants of interest include vegetables, oil-seed plants, leguminous plants (e.g. Fabaceae/Leguminosae family), ornamentals, and conifers. Plants of interest include for example soybean (Glycine max), corn (Zea mays), Brassica spp. (e.g., B. napus, B. rapa, B. juncea), other Brassicaceae family (e.g. Arabidopsis spp.), alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), Cannabaceae family (e.g., Cannabis spp., e.g. Cannabis sativa), foxtail millet (Setaria italica), finger millet (Eleusine coracana)), sunflower (Helianthus annuus), safflower (Carthamus tinctorius), wheat (Triticum aestivum), tobacco (Nicotiana tabacum), potato (Solanum tuberosum), peanuts (Arachis hypogaea), cotton (Gossypium barbadense, Gossypium hirsutum), sweet potato (Ipomoea batatus), Allium spp. (onion, shallot, chives, leek), carrot (Daucus spp., e.g. Daucus carota), grapevines (Vitis spp.) cassaya (Manihot esculenta), coffee (Coffea spp.), coconut (Cocos nucifera), pineapple (Ananas comosus), citrus trees (Citrus spp.), apple (Malus spp.) cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers. Vegetables include tomatoes (Lycopersicon lycopersicon), lettuce (e.g., Lactuca sativa), green beans (Phaseolus vulgaris), lima beans (Phaseolus limensis), peas (Lathyrus spp.), Cucumis spp. such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicate) and Alaska yellow-cedar (Chamaecyparis nootkatensis). In an embodiment, the plant is of the family Solanaceae, in a further embodiment of the genus Solanum. In embodiments, the plants are crop plants (for example, potato, onion, carrot, grapevines, corn, alfalfa, sunflower, Brassica, soybean, cotton, safflower, peanut, sorghum, wheat, millet, tobacco, cannabis, etc.). In an embodiment, the plant is soybean (Glycine max). In embodiments, the plant is can be used in the production of food crops, biofuels, biomass, medicinals and animal feed.

### EXAMPLES

The present disclosure is illustrated in further detail by the following non-limiting examples.

### Example 1: Bacteria Isolation and Culture Preparation

Bacteria were isolated from root nodules of a perennial legume, Amphicarpaea bracteata (American Hog peanut) which is indigenous to Canada and the U.S.A. It grows in damp bottomlands, riparian woods and thickets and their roots and seeds are edible. The nodules collected were surface sterilized and contents were serially diluted and plated on Kings B and yeast extract mannitol (YEM) agar plates. The plates were incubated at 28° C. and distinct colonies were isolated. The isolates were identified by 16s rRNA sequencing and stored in glycerol stocks for further use. Bacteria were grown for 24-48 h (until the log phase) at 28° C. and the cultures were harvested. The inoculum was suspended in 10 mM MgSO₄, adjusted to 0.5 O.D at A_{600 nm}.

### Example 2: Seed Germination Assay

Soybean seeds (Absolute RR) were treated with bacterial suspension in for 30 min and then placed on Petri dishes lined with filter paper. The filter paper discs were previously saturated with water or 100 mM NaCl solution. The plates were then incubated in dark at 25° C. Germination was observed at 24, 36 and 48 h after the seeds were placed in the Petri plates. Two bacteria (AB2 and AB8) significantly increased germination rate (>60%) when compared to the control and among the 30 isolates screened. (FIG. 2).

### Example 3: Plant Growth Experiments

### Preliminary Screening for Plant Growth Promoting Activity:

Bacterized seeds were placed on 15.25 cm pots filled with vermiculite previously moistened with water or 100 mM NaCl in water. There were six pots (replicates) of each treatment, organized in a randomized complete block design (RCBD). Seedling emergence was counted after one week and plants were harvested after three weeks. Vegetative growth variables, including plant height, leaf area, shoot and root biomass were measured.

### Inducing Salt Tolerance:

Two isolates selected from the preliminary experiment were tested for inducing stress tolerance under a range of salt concentrations (0 to 200 mM) in the greenhouse. Soybean seeds were treated with bacteria and the plants were sampled after 4 weeks. Vegetative growth variables, including plant height, leaf area, shoot and root biomass were measured.

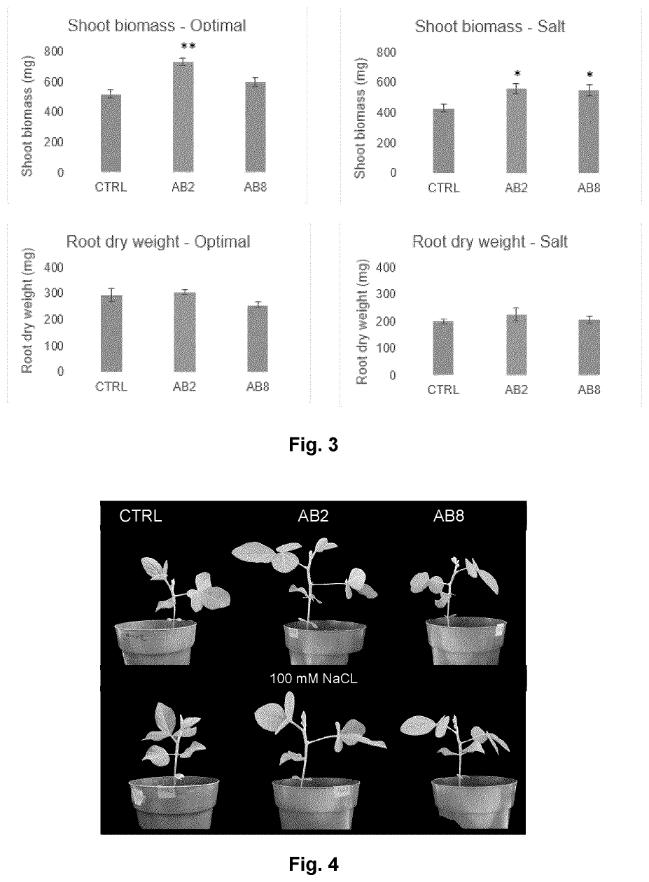

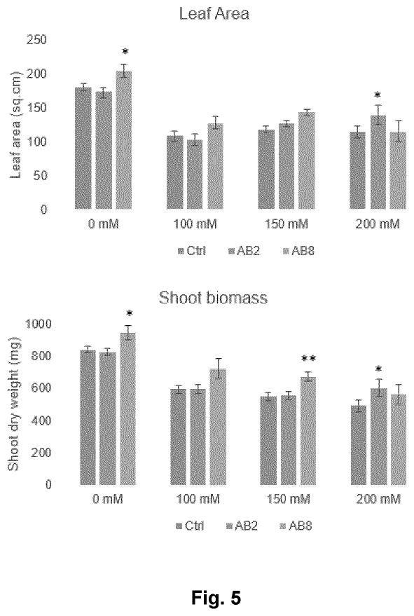

Salt stress tolerance induced by the PGPR strains AB2 and AB8 was confirmed as they significantly improved soybean growth and development under salt stress and increased shoot biomass by 28.2% and 27.5%, respectively (FIG. 3). These strains also increased the growth of unstressed soybean plants (FIG. 4).

Soybean plants treated with AB2 and AB8 showed improved plant growth with increase in leaf area and shoot biomass under different concentrations of salt concentrations (FIG. 5.)

### Example 4: Arabidopsis Root Growth—In Vitro

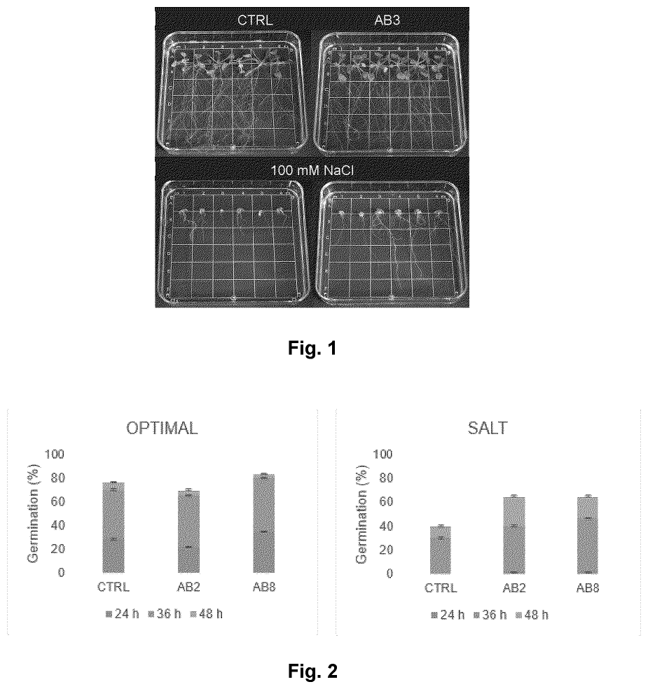

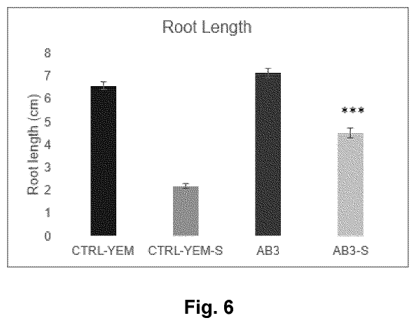

Supernatants of bacterial cultures were filter sterilized and added to ½ MS media. Stratified Arabidopsis seeds were placed on the media and incubated at 22° C. for two weeks. Clear differences in root growth were observed in Arabidopsis under salt stress in treatment with the supernatant from isolate AB3, which resulted in a greater than 100% increase in root length when compared to control (FIGS. 1 and 6).

### Example 5: Seed Germination Studies with Cell Free Supernatant

Seed germination experiments were conducted in order to assess the efficacy of the cell free supernatant (CFS) from isolate AB3.

The products were tested on different crops, indicated below.

For canola, CFS treatment increased the final seed germination by around 1.5% as compare to control. Results are shown in Table 1. CFS was used at a dilution of 1:1000. Control treatment was with water.

TABLE 1
Canola seed germination

| Treatments | 18 h | ±SE | 24 h | ±SE | 30 h | ±SE |
|---|---|---|---|---|---|---|
| p-value | 0.0003 | | 0.02 | | 0.04 | |
| Control | 66.25 | 2.3 | 90 | 5.4 | 95 | 2.04 |
| CFS | 65 | 2.04 | 87.5 | 1.4 | 96.25 | 2.3 |

For soybean, CFS treatment also showed an increase relative to control, with 100% seed germination and an increase in germination by 8% relative to control. Results are shown in Table 2. CFS was used at a dilution of 1:1000. Control treatment was with water.

TABLE 2
Soybean seed germination

| Treatments | 24 h | ±SE | 48 h | ±SE | 72 h | ±SE |
|---|---|---|---|---|---|---|
| p-value | 0.6436 | | 0.1366 | | 0.4 | |
| Control | 42.5 | 4.7 | 90 | 0 | 92.5 | 2.5 |
| CFS | 55 | 9.57 | 100 | 0 | 100 | 0 |

For corn, CFS treatment increased the rate of seed germination by 15%, with a 5% increase in germination. Results are shown in Table 3. CFS was used at a dilution of 1:1000. Control treatment was with water.

TABLE 3
Corn seed germination

| | | | | |
|---|---|---|---|---|
| Treatments | 72 h | ±SE | 96 h | ±SE |

| | | |
|---|---|---|
| p-value | 0.4320 | <.0001 |

| | | | | |
|---|---|---|---|---|
| Control | 55 | 6.4 | 95 | 2.8 |
| CFS | 70 | 4.08 | 100 | 0 |

Further germination studies were carried out to assess the efficacy of the cell free supernatant (CFS) from isolate AB3 on chili pepper, Chinese celery, eggplant and tomato, with results shown in Tables 4-7 below. Sup=CFS, used at the dilutions indicated below. Control is water.

TABLE 4
Chili pepper

| Treatments | Day 4 | ±SE | Day 6 | ±SE | Day 7 | ±SE | Day 8 | ±SE | Day 9 | ±SE | Day 10 | ±SE |
|---|---|---|---|---|---|---|---|---|---|---|---|---|
| p-value | 0.3405 | | 0.0003 | | 0.0277 | | 0.2983 | | 0.4914 | | 0.4359 | |
| Control | 4.00 | 1.00 | 67.00 | 4.64 | 83.00 | 7.35 | 92.00 | 3.00 | 91.00 | 2.92 | 94.00 | 2.92 |
| Sup 1:100 | 5.00 | 1.58 | 65.00 | 6.89 | 81.00 | 4.00 | 93.00 | 2.00 | 95.00 | 1.58 | 95.00 | 1.58 |
| Sup 1:250 | 6.00 | 3.67 | 65.00 | 3.54 | 87.00 | 2.55 | 93.00 | 3.00 | 95.00 | 1.58 | 99.00 | 1.00 |
| Sup 1:500 | 11.00 | 2.92 | 72.00 | 2.55 | 88.00 | 2.55 | 95.00 | 3.16 | 98.00 | 2.00 | 99.00 | 1.00 |
| Sup 1:750 | 10.00 | 2.74 | 80.00 | 5.24 | 92.00 | 2.55 | 97.00 | 1.22 | 98.00 | 1.22 | 100.00 | 0.00 |
| Sup 1:1000 | 5.00 | 0.00 | 63.00 | 3.74 | 82.00 | 2.00 | 88.00 | 1.22 | 91.00 | 1.87 | 95.00 | 2.24 |

TABLE 5
Chinese Celery

| Treatments | Day 3 | ±SE | Day 4 | ±SE | Day 6 | ±SE | Day 7 | ±SE | Day 8 | ±SE | Day 9 | ±SE | Day 10 | ±SE | Day 14 | ±SE |
|---|---|---|---|---|---|---|---|---|---|---|---|---|---|---|---|---|
| p-value | 0.0114 | | <0.0001 | | <0.0001 | | 0.0024 | | 0.0325 | | 0.0329 | | 0.0565 | | 0.5870 | |
| Control | 3.00 | 1.22 | 18.00 | 2.55 | 57.00 | 6.44 | 61.00 | 4.30 | 62.00 | 6.04 | 61.00 | 5.34 | 62.00 | 5.15 | 67.00 | 6.04 |
| Sup 1:100 | 1.00 | 1.00 | 11.00 | 4.00 | 48.00 | 4.64 | 68.00 | 2.55 | 61.00 | 5.79 | 65.00 | 5.24 | 68.00 | 6.04 | 66.00 | 7.65 |
| Sup 1:250 | 3.00 | 1.22 | 12.00 | 2.55 | 54.00 | 4.00 | 62.00 | 5.83 | 63.00 | 3.74 | 60.00 | 2.74 | 63.00 | 3.39 | 67.00 | 4.36 |
| Sup 1:500 | 1.00 | 1.00 | 10.00 | 3.54 | 60.00 | 4.47 | 68.00 | 5.83 | 67.00 | 2.55 | 68.00 | 3.39 | 68.00 | 3.39 | 68.00 | 5.15 |
| Sup 1:750 | 0.00 | 0.00 | 18.00 | 4.36 | 49.00 | 2.92 | 62.00 | 1.22 | 55.00 | 4.18 | 58.00 | 4.36 | 60.00 | 3.54 | 65.00 | 5.70 |
| Sup 1:1000 | 0.00 | 0.00 | 15.00 | 3.54 | 70.00 | 3.54 | 77.00 | 2.00 | 75.00 | 1.58 | 74.00 | 1.87 | 75.00 | 1.58 | 73.00 | 2.55 |

TABLE 6
Eggplant

| Treatments | Day 3 | ±SE | Day 4 | ±SE | Day 6 | ±SE | Day 7 | ±SE | Day 8 | ±SE | Day 9 | ±SE | Day 10 | ±SE |
|---|---|---|---|---|---|---|---|---|---|---|---|---|---|---|
| p-value | <0.0001 | | 0.0337 | | <0.0001 | | <0.0001 | | <0.0001 | | <0.0001 | | <0.0001 | |
| Control | 0.00 | 0.00 | 11.00 | 4.85 | 34.00 | 4.30 | 48.00 | 5.15 | 68.00 | 4.06 | 77.00 | 4.90 | 78.00 | 4.90 |
| Sup 1:100 | 5.00 | 1.58 | 15.00 | 6.32 | 46.00 | 6.20 | 60.00 | 4.47 | 74.00 | 4.30 | 80.00 | 3.16 | 82.00 | 4.06 |
| Sup 1:250 | 0.00 | 0.00 | 3.00 | 2.00 | 23.00 | 3.39 | 36.00 | 2.45 | 53.00 | 5.15 | 58.00 | 5.39 | 58.00 | 5.39 |
| Sup 1:500 | 2.00 | 1.22 | 10.00 | 5.24 | 45.00 | 4.18 | 51.00 | 6.78 | 72.00 | 5.39 | 78.00 | 2.00 | 84.00 | 3.67 |
| Sup 1:750 | 6.00 | 1.87 | 23.00 | 3.39 | 53.00 | 6.63 | 63.00 | 3.74 | 74.00 | 6.20 | 80.00 | 5.70 | 85.00 | 3.16 |
| Sup 1:1000 | 0.00 | 0.00 | 5.00 | 1.58 | 41.00 | 4.00 | 59.00 | 2.45 | 69.00 | 5.10 | 79.00 | 3.67 | 83.00 | 2.55 |

TABLE 7
Tomato

| Treatments | Day 6 | ±SE | Day 7 | ±SE | Day 8 | ±SE | Day 9 | ±SE | Day 10 | ±SE | Day 14 | ±SE |
|---|---|---|---|---|---|---|---|---|---|---|---|---|
| p-value | <0.0001 | | <0.0001 | | <0.0001 | | 0.0136 | | 0.0310 | | 0.0329 | |
| Control | 26.00 | 4.00 | 47.00 | 6.24 | 60.00 | 2.74 | 66.00 | 2.45 | 70.00 | 3.16 | 68.00 | 3.39 |
| Sup 1:100 | 33.00 | 4.64 | 53.00 | 3.00 | 65.00 | 0.00 | 67.00 | 1.22 | 74.00 | 1.87 | 76.00 | 2.92 |
| Sup 1:250 | 21.00 | 1.87 | 49.00 | 5.57 | 63.00 | 5.39 | 73.00 | 5.15 | 77.00 | 5.61 | 77.00 | 4.64 |
| Sup 1:500 | 32.00 | 6.44 | 48.00 | 6.44 | 67.00 | 3.00 | 76.00 | 4.00 | 78.00 | 4.36 | 74.00 | 4.00 |
| Sup 1:750 | 22.00 | 1.22 | 53.00 | 1.22 | 62.00 | 4.64 | 63.00 | 4.36 | 70.00 | 4.18 | 69.00 | 4.58 |
| Sup 1:1000 | 30.00 | 7.58 | 65.00 | 7.07 | 75.00 | 2.24 | 78.00 | 2.55 | 82.00 | 2.00 | 85.00 | 1.58 |

This project has allowed the identification and characterization of new PGPR strains isolated from native legume species. The ability to increase plant growth and biomass production, under both stressed and unstressed conditions, has been shown for isolates described herein. It has also been shown that cell free supernatants have growth promotion properties, i.e. the presence of active compounds in the crude extract having such growth promoting activity, which will lead to the development of novel growth promoting substances. These novel PGPR strains, or products/extracts/substantially cell free supernatants thereof, can be effectively applied as an agriculture input to improve soybean, and a wide range of crops, including biomass crops, to improve productivity, for example under stress conditions.

The following biological material has been deposited under the terms of the Budapest Treaty at the International Depository Authority of Canada (IDAC), National Microbiology Laboratory of Canada, 1015 Arlington Street, Winnipeg, Manitoba, R3E 3R2:

| Bacterial strain | Accession No. | Date of Deposit |
|---|---|---|
| AB2 | 090719-01 | Jul. 9, 2019 |
| AB3 | 090719-02 | Jul. 9, 2019 |
| AB8 | 090719-03 | Jul. 9, 2019 |

Although the present disclosure has been described hereinabove by way of specific embodiments thereof, it can be modified, without departing from the spirit and nature of the subject disclosure as defined in the appended claims.

### REFERENCES

1. Wang N, Khan W, Smith D L 2012. Soybean global gene expression after application of lipo-chitooligosaccharide from Bradyrhizobium japonicum under sub-optimal temperature. PLoS ONE 7(2): e31571. doi:10.1371/journal.pone.0031571.
2. Lee K D, Gray E J, Mabood F, Jung W J, Charles T, Clark S R D, Ly A, Souleimanov A, Zhou X, Smith D L 2009. The class IId bacteriocin thuricin 17 increases plant growth. Planta 229:747-755.
3. Almaraz J J, Mabood F, Zhou X, Gregorich E G and Smith D L 2008. Climate change, weather variability and corn yield at a higher latitude locale: southwestern Quebec. Climatic Change 88:187-197.
4. Almaraz J, Zhou X and Smith D L 2007. Gas exchange characteristics and dry matter accumulation of soybean treated with Nod factors. J Plant Phys 164:1391-1393.
5. Mabood F, Souleimanov A, Khan W and Smith D L 2006. Jasmonates induce Nod factor production by Bradyrhizobium japonicum. Plant Physiol Biochem 44:759-765.
6. Gray E, Di Falco M, Souleimanov A and Smith D L 2006. Proteomic analysis of the bacteriocin, thuricin 17 produced by Bacillus thuringiensis NEB17. FEMS Microbiology Letters 255:27-32.
7. Mabood F, Zhou X, Lee K D, Smith D L 2006. Methyl jasmonate, alone or in combination with genistein, and Bradyrhizobium japonicum increases soybean (Glycine max L.) plant dry matter production and grain yield under short season conditions. Field Crops Research 95:412-419.
8. Mabood F and Smith D L 2005. Pre-incubation of Bradyrhizobium japonicum with jasmonates accelerates nodulation and nitrogen fixation in soybean (Glycine max) at optimal and suboptimal root zone temperatures. Physiologia Plantarum 125:311-325.
9. Gray, E. J. and Smith, D. L. 2005. Intracellular and Extracellular PGPR: Commonalities and distinctions in the plant-bacterium signaling processes. Soil Biol Biochem 37:395-412.
10. Smith, D. L. and Almaraz, J. J. 2004. Climate change and crop production: Contributions, impacts and adaptations. Can J Plant Pathol 26: 253-266.

What is claimed is:

1. A biologically pure culture of a bacterial strain selected from a bacterial strain comprising substantially all of the biochemical characteristics of bacterial strain AB2 deposited at the IDAC under accession no. 090719-01 on Jul. 9, 2019 or a mutant thereof comprising substantially all of the biochemical characteristics thereof; a bacterial strain comprising substantially all of the biochemical characteristics of bacterial strain AB3 deposited at the IDAC under accession no. 090719-02 on Jul. 9, 2019 or a mutant thereof comprising substantially all of the biochemical characteristics thereof; and a bacterial strain comprising substantially all of the biochemical characteristics of bacterial strain AB8 deposited at the IDAC under accession no. 090719-03 on Jul. 9, 2019 or a mutant thereof comprising substantially all of the biochemical characteristics thereof.

2. The biologically pure culture of a bacterial strain of claim 1, wherein the bacterial strain is a bacterial strain comprising substantially all of the biochemical characteristics of bacterial strain AB2 deposited at the IDAC under accession no. 090719-01 on Jul. 9, 2019 or a mutant thereof comprising substantially all of the biochemical characteristics thereof.

3. The biologically pure culture of a bacterial strain of claim 1, wherein the bacterial strain is a bacterial strain comprising substantially all of the biochemical characteristics of bacterial strain AB3 deposited at the IDAC under accession no. 090719-02 on Jul. 9, 2019 or a mutant thereof comprising substantially all of the biochemical characteristics thereof.

4. The biologically pure culture of a bacterial strain of claim 1, wherein the bacterial strain is a bacterial strain comprising substantially all of the biochemical characteristics of bacterial strain AB8 deposited at the IDAC under accession no. 090719-03 on Jul. 9, 2019 or a mutant thereof comprising substantially all of the biochemical characteristics thereof.

5. A biologically pure culture of a bacterial strain selected from bacterial strain AB2 deposited at the IDAC under accession no. 090719-01 on Jul. 9, 2019 or a mutant thereof comprising substantially all of the biochemical characteristics thereof; bacterial strain AB3 deposited at the IDAC under accession no. 090719-02 on Jul. 9, 2019 or a mutant thereof comprising substantially all of the biochemical characteristics thereof; and bacterial strain AB8 deposited at the IDAC under accession no. 090719-03 on Jul. 9, 2019 or a mutant thereof comprising substantially all of the biochemical characteristics thereof.

6. The biologically pure culture of a bacterial strain of claim 5, wherein the bacterial strain is bacterial strain AB2 deposited at the IDAC under accession no. 090719-01 on Jul. 9, 2019 or a mutant thereof comprising substantially all of the biochemical characteristics thereof.

7. The biologically pure culture of a bacterial strain of claim 5, wherein the bacterial strain is bacterial strain AB3 deposited at the IDAC under accession no. 090719-02 on Jul. 9, 2019 or a mutant thereof comprising substantially all of the biochemical characteristics thereof.

8. The biologically pure culture of a bacterial strain of claim 5, wherein the bacterial strain bacterial strain AB8 deposited at the IDAC under accession no. 090719-03 on Jul. 9, 2019 or a mutant thereof comprising substantially all of the biochemical characteristics thereof.

9. A culture product of the bacterial strain defined in any one of claims 1 to 8.

10. A composition comprising the bacterial strain defined in any one of claims 1 to 8 and/or the culture product of claim 9 and a carrier.

11. A method comprising applying the bacterial strain defined in any one of claims 1 to 8, the culture product of claim 9, and/or the composition of claim 10, to a plant or part thereof, a seed of a plant, and/or an area around the seed, plant or part thereof.

12. A method for increasing a plant's growth, comprising applying the bacterial strain defined in any one of claims 1 to 8, the culture product of claim 9, and/or the composition of claim 10, to a plant or part thereof, a seed of a plant, and/or an area around the seed, plant or part thereof, in an amount effective to produce an increase in plant growth as compared to the growth of the plant in the absence of said application of said bacterial strain, culture product and/or composition.

13. The method of claim 11 or 12, wherein the plant or seed is for growth under abiotic stress conditions.

14. The method of claim 13, wherein the abiotic stress conditions are selected from high salinity, drought, high temperature, low temperature and flooding.

15. The method of claim 14, wherein the abiotic stress conditions comprise high salinity.

16. A method for increasing tolerance of a plant or seed to one or more abiotic stress conditions, comprising applying the bacterial strain defined in any one of claims 1 to 8, the culture product of claim 9, and/or the composition of claim 10, to the plant or part thereof, the seed, and/or an area around the seed, plant or part thereof, in an amount effective to produce an increase in tolerance to the one or more abiotic stress conditions as compared to the tolerance of the plant in the absence of said application of said bacterial strain, culture product and/or composition.

17. The method of claim 16, wherein the abiotic stress conditions are selected from high salinity, drought, high temperature, low temperature and flooding.

18. The method of claim 17, wherein the abiotic stress conditions comprise high salinity.

19. The method of any one of claims 11 to 18, wherein the plant is a leguminous plant.

20. The method of claim 19, wherein the plant is soybean.

21. The composition of claim 10, which is a seed coating composition.

22. A seed partially or completely coated with the bacterial strain defined in any one of claims 1 to 8, the culture product of claim 9, and/or the composition of claim 10 or 21.

23. A method of preparing the culture product of claim 9, comprising culturing the bacterial strain defined in any one of claims 1 to 8 and recovering the culture product from the culture.

24. A kit comprising the bacterial strain defined in any one of claims 1 to 8, the culture product of claim 9, and/or the composition of claim 10.

25. The kit of claim 24, further comprising instructions for use of the bacterial strain, culture product, and/or composition.
